(19) United States
(12) Patent Application Publication
(10) Pub. No.: US 2022/0362496 A1
(43) Pub. Date: Nov. 17, 2022

(54) METHOD FOR CONTROLLING OXYGEN-CONTAINING GAS AND RELATED PRODUCTS

(71) Applicant: TELESAIR, INC., Irvine, CA (US)

(72) Inventor: QING WANG, PALO ALTO, CA (US)

(21) Appl. No.: 17/321,151

(22) Filed: May 14, 2021

(51) Int. Cl. A61M 16/00; A61M 16/06

(52) U.S. Cl. CPC A61M 16/024; A61M 16/0672; A61M 2016/0027; A61M 2016/003; A61M 2205/52; A61M 2202/0208; A61M 16/0066

(57) ABSTRACT

Provided are a method for controlling oxygen-containing gas output by an oxygen provider, The method includes: acquiring a first pressure measurement of the oxygen-containing gas at the oxygen provider side; determining a pressure estimation of the oxygen-containing gas at the patient side based on the first pressure measurement, wherein the oxygen-containing gas experiences a pressure drop on the gas pathway to arrive at the patient side; and controlling a target parameter of the oxygen-containing gas output by the oxygen provider based on the pressure estimation. With the method or device for controlling oxygen-containing gas output by an oxygen provider, an automated solution to control the oxygen-containing gas based on a pressure measurement at the oxygen provider side is provided, and the complexity of a medical device for oxygen therapy is reduced.

### TECHNICAL FIELD

The present disclosure relates generally to the technical field of medical devices for oxygen therapy and, in particular, to a method for dynamically controlling an oxygen provider a controlling device, and a computer readable storage medium.

### BACKGROUND

A respiratory disease often reduces a patient's oxygen level in blood, resulting in hypoxemia, a condition that damages heart, brain, and other human organs. Oxygen therapy is widely used in medical treatment to patients with respiratory diseases.

A respiratory disease may be caused by viruses, for example, the COVID-19 virus. In the current COVID-19 pandemic, oxygen therapy is highly recommended for treatment by WHO (World Health Organization) and healthcare authorizations including National Institute of Health of the United States.

This background information is provided to reveal information believed by the applicant to be of possible relevance to the present disclosure. No admission is necessarily intended, nor should be construed, that any of the preceding information constitutes prior art against the present disclosure.

### SUMMARY

The present disclosure provides a method for controlling an oxygen provider and related products.

A first aspect of the present disclosure relates to a method for controlling oxygen-containing gas output by an oxygen provider, the oxygen provider being capable of delivering the oxygen-containing gas via a gas pathway from the oxygen provider side to a patient side, the method includes:

acquiring a first pressure measurement of the oxygen-containing gas at the oxygen provider side;

determining a pressure estimation of the oxygen-containing gas at the patient side based on the first pressure measurement, where the oxygen-containing gas experiences a pressure drop on the gas pathway to arrive at the patient side; and controlling a target parameter of the oxygen-containing gas output by the oxygen provider based on the pressure estimation.

In a possible implementation form of the method according to the first aspect as such, where before the determining a pressure estimation of the oxygen-containing gas at the patient side based on the first pressure measurement, the method further includes:

determining a characteristic property of the gas pathway from the oxygen provider side to the patient side;

where the determining a pressure estimation of the oxygen-containing gas at the patient side based on the first pressure measurement, includes:

acquiring a first flow measurement at the oxygen provider side; and

determining the pressure estimation based on the characteristic property, the first flow measurement and the first pressure measurement.

In a possible implementation form of the method according to the first aspect as such, where the determining a characteristic property of the gas pathway from the oxygen provider side to the patient side, includes:

acquiring a set of second pressure measurements at the oxygen provider side and a set of second flow measurements at the oxygen provider side in a case where a pressure at the patient side is zero; and determining the characteristic property based on the set of second pressure measurements and the set of second flow measurements.

In a possible implementation form of the method according to the first aspect as such, where the determining the pressure estimation based on the characteristic property, the first flow measurement and the first pressure measurement, includes:

determining the pressure drop based on the characteristic property and the first flow measurement; and

determining the pressure estimation based on the first pressure measurement and the pressure drop.

In a possible implementation form of the method according to the first aspect as such, where the target parameter of the oxygen-containing gas includes a flow rate of the oxygen-containing gas, the controlling a target parameter of the oxygen-containing gas output by the oxygen provider based on the pressure estimation, includes:

determining a flow setting of the oxygen provider based on the pressure estimation; and

controlling the flow rate of the oxygen-containing gas output by the oxygen provider based on the flow setting of the oxygen provider.

In a possible implementation form of the method according to the first aspect as such, where the determining a pressure estimation of the oxygen-containing gas at the patient side based on the first pressure measurement, includes:

determining multiple pressure estimations of the oxygen-containing gas in a first time span;

where before the controlling the flow rate of the oxygen-containing gas output by the oxygen provider based on the flow setting of the oxygen provider, the method further includes:

calculating a mean pressure of the determined multiple pressure estimations;

where the determining a flow setting of the oxygen provider based on the pressure estimation, includes:

in a second time span following the first time span, increasing the flow setting of the oxygen provider in response to that the mean pressure is lower than a desired pressure; or

in the second time span following the first time span, decreasing the flow setting of the oxygen provider in response to that the mean pressure is higher than the desired pressure.

A second aspect of the present disclosure relates to a controlling device, including:

a detecting module, configured to acquire a first pressure measurement of the oxygen-containing gas at the oxygen provider side;

a processing module, configured to determine a pressure estimation of the oxygen-containing gas at the patient side based on the first pressure measurement, where the oxygen-containing gas experiences a pressure drop on the gas pathway to arrive at the patient side; and

a controlling module, configured to control a target parameter of the oxygen-containing gas output by the oxygen provider based on the pressure estimation.

In a possible implementation form of the controlling device according to the second aspect as such, where,

the processing module is further configured to determine a characteristic property of the gas pathway from the oxygen provider side to the patient side;

the detecting module is further configured to acquire a first flow measurement at the oxygen provider side; and

the processing module is further configured to determine the pressure estimation based on the characteristic property, the first flow measurement and the first pressure measurement.

In a possible implementation form of the controlling device according to the second aspect as such, where,

the detecting module is further configured to acquire a set of second pressure measurements at the oxygen provider side and a set of second flow measurements at the oxygen provider side in a case where a pressure at the patient side is zero; and

the processing module is further configured to determine the characteristic property based on the set of second pressure measurements and the set of second flow measurements.

In a possible implementation form of the method according to the first aspect as such, where,

the processing module is further configured to determine the pressure drop based on the characteristic property and the first flow measurement; and determine the pressure estimation based on the first pressure measurement and the pressure drop.

In a possible implementation form of the controlling device according to the second aspect as such, where,

the processing module is further configured to determine a flow setting of the oxygen provider based on the pressure estimation; and

the controlling module is further configured to control the flow rate of the oxygen-containing gas output by the oxygen provider based on the flow setting of the oxygen provider.

In a possible implementation form of the controlling device according to the second aspect as such, where the processing module is configured to:

determine multiple pressure estimations of the oxygen-containing gas in a first time span; calculate a mean pressure of the determined multiple pressure estimations; and

in a second time span following the first time span, increase the flow setting of the oxygen provider in response to that the mean pressure is lower than a desired pressure; or

in the second time span following the first time span, decrease the flow setting of the oxygen provider in response to that the mean pressure is higher than the desired pressure.

A third aspect of the present disclosure relates to a controlling device, the controlling device is communicatively connected to an oxygen provider, the oxygen provider being capable of delivering the oxygen-containing gas via a gas pathway from the oxygen provider side to a patient side, and the controlling device includes:

at least one processor; and

a memory communicatively connected with the at least one processor; where,

the memory stores instructions executable by the at least one processor, and the instructions, when executed by the at least one processor, cause the at least one processor to implement the method for controlling an oxygen provider according to the first aspect and the possible implementation forms.

A fourth aspect of the present disclosure relates to a high flow nasal cannula (HFNC) device, the HFNC device is communicatively connected to an oxygen provider, the oxygen provider being capable of delivering the oxygen-containing gas via a gas pathway from the oxygen provider side to a patient side, where the HFNC device includes the controlling device according to the second aspect and the possible implementation forms.

A fifth aspect of the present disclosure relates to a computer readable storage medium, storing thereon computer executable instructions which, when being executed by a processor, implement the method for controlling an oxygen provider according to the first aspect and the possible implementation forms.

### BRIEF DESCRIPTION OF DRAWINGS

FIG. 1 is a schematic diagram of an oxygen delivering system according to an embodiment of the present disclosure;

FIG. 2 is a schematic flowchart of a method for controlling oxygen-containing gas output by an oxygen provider according to an embodiment of the present disclosure;

FIG. 3 is a schematic flowchart of a method for controlling oxygen-containing gas output by an oxygen provider according to an embodiment of the present disclosure;

FIG. 4 is a schematic illustration of time span arrangement according to an embodiment of the present disclosure;

FIG. 5 is a schematic diagram for updating the flow setting of the oxygen provider according to an embodiment of the present disclosure;

FIG. 6 is a schematic structural diagram of a controlling device according to an embodiment of the present disclosure;

FIG. 7 is a schematic structural diagram of a controlling device according to an embodiment of the present disclosure;

FIG. 8 is a schematic diagram of a high flow nasal cannula device according to an embodiment of the present disclosure; and

FIG. 9 is a schematic diagram of a high flow nasal cannula device according to an embodiment of the present disclosure.

### DESCRIPTION OF EMBODIMENTS

In the following description, reference is made to the accompanying figures, which form part of the disclosure, and which show, by way of illustration, specific aspects of embodiments of the present disclosure or specific aspects in which embodiments of the present disclosure may be used. It is understood that embodiments of the present disclosure may be used in other aspects and comprise structural or logical changes not depicted in the figures. The following detailed description, therefore, is not to be taken in a limiting sense, and the scope of the present disclosure is defined by the appended claims.

For instance, it is understood that a disclosure in connection with a described method may also hold true for a corresponding device or system configured to perform the method and vice versa. For example, if one or a plurality of specific method steps are described, a corresponding device may include one or a plurality of units, e.g. functional units, to perform the described one or plurality of method steps (e.g. one unit performing the one or plurality of steps, or a plurality of units each performing one or more of the plurality of steps), even if such one or more units are not explicitly described or illustrated in the figures. On the other hand, for example, if a specific apparatus is described based on one or a plurality of units, e.g. functional units, a corresponding method may include one step to perform the functionality of the one or plurality of units (e.g. one step performing the functionality of the one or plurality of units, or a plurality of steps each performing the functionality of one or more of the plurality of units), even if such one or plurality of steps are not explicitly described or illustrated in the figures. Further, it is understood that the features of the various exemplary embodiments and/or aspects described herein may be combined with each other, unless specifically noted otherwise.

In the embodiments of the present disclosure, expressions such as “exemplary” or “for example” are used to indicate illustration of an example or an instance. In the embodiments of the present disclosure, any embodiment or design scheme described as “exemplary” or “for example” should not be interpreted as preferred or advantageous over other embodiments or design schemes. In particular, the use of “exemplary” or “for example” is aimed at presenting related concepts in a specific manner.

Several terms that may be used in the present disclosure are briefly explained herein.

Oxygen, which refers to pure oxygen or high concentration oxygen (95% or above) that is used in oxygen therapy.

Oxygen-containing gas, which refers to a mixture of oxygen and air for delivering to a patient. The proportion of oxygen in the oxygen-containing gas is not specifically limited in the present disclosure.

Gas is used herein as a general reference to oxygen, air or oxygen-containing gas.

An oxygen source, which refers to a piece of medical equipment that provides oxygen, including but not limited to a cylinder, a concentrator, an oxygen plant or a liquid oxygen supplier. Where, a cylinder is a refillable cylindrical storage vessel used to store and transport oxygen in compressed gas form; a concentrator is a self-contained, electrically powered medical device designed to concentrate oxygen from ambient air; an oxygen plant is an onsite oxygen generating system using pressure swing adsorption (PSA) which serves as a large, central source of oxygen generation, and can be located on-site at medical facilities; a liquid oxygen supplier contains bulk liquid oxygen generated offsite and stored in a large tank and supplied throughout a health facility pipeline system, and the tank requires refilling by liquid oxygen supplier.

An oxygen provider, which is a device or a system that provides oxygen-containing gas. For example, an oxygen provider may include a low-pressure oxygen source which generates low-pressure oxygen, an accumulator where the low-pressure oxygen and ambient air are mixed to produce the oxygen-containing gas, and a blower device to blow the oxygen-containing gas to a patient. The low-pressure oxygen source may be a concentrator which concentrates the oxygen from ambient air by removing nitrogen selectively to create a high-concentration oxygen gas stream, or a cylinder and a flow regulator, where the cylinder provides high-pressure oxygen and the flow regulator regulates compressed high-pressure oxygen to low-pressure oxygen, or a liquid oxygen supplier and a flow regulator which regulates liquid oxygen to low-pressure oxygen (in gas form).

In oxygen therapy, oxygen provided by an oxygen source is often mixed with air, so as to produce oxygen-containing gas suitable to achieve a therapeutic fraction ratio of inspired oxygen (FiO2). The oxygen-containing gas of a certain FiO2 is delivered to a patient via an airway tube and a patient interface, for example, a nasal cannula, a mask, or a tracheostomy adaptor.

The oxygen containing gas delivered to a patient is often manually controlled by a caregiver. For example, in conventional oxygen therapy, FiO2 of the oxygen containing gas, or a flow rate of the oxygen containing gas are manually set by a caregiver empirically.

In terms of patients infected by contagious virus such as COVID-19, a manual solution will expose a caregiver to risks of being infected, and add workload to a caregiver.

Thus, an automated solution to control the oxygen-containing gas for delivering to a patient is desired.

A method for controlling oxygen-containing gas output by an oxygen provider is described in the present disclosure. An aim of the solution provided in the present disclosure is to control the oxygen-containing gas output by an oxygen provider in an automated manner.

In conventional oxygen therapy, a caregiver, for example, a nurse or a doctor, sets a medical device for oxygen therapy manually based on some parameters measured from a patient. The measured parameters may include but not limited to a blood pressure, a heart rate, a body temperature, an arterial hemoglobin oxygen saturation (SpO₂) signal, an airway pressure, a chest movement range of the patient etc.

These parameters are measured through various sensors. For example, in high flow nasal cannula (HFNC) oxygen therapy, a positive pressure is required to be maintained in a nasal cavity or a respiratory tract of a patient, which is helpful in carbon dioxide (CO2) clearance, Oxygen (O2) exchange, therefore, in conventional HFNC oxygen therapy, a pressure sensor is integrated in a proximal pressure line from user interface for measuring the pressure.

However, the proximal pressure line is cumbersome and hard to manage. In a practical application scenario, the proximal pressure line can be easily twisted, or blocked by moisture in exhalation of a patient. Therefore, the solution described in this disclosure is also aimed at reducing the complexity of a medical device for oxygen therapy.

In the solution described herein, a parameter at a patient side is estimated based on a parameter measured at an oxygen provider side. That is, the conventional measurement at the patient side is transferred to the provider side. Therefore, a sensor for measuring the parameter can be connected at the oxygen provider side, or integrated in the oxygen provider, in this way the proximal pressure line in the conventional HFNC oxygen therapy is no longer required, thereby reducing the complexity of a medical device for oxygen therapy.

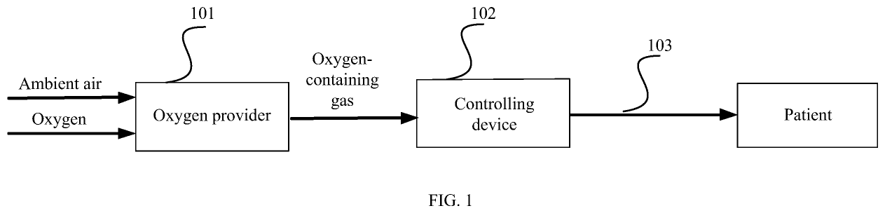

FIG. 1 is an exemplary schematic diagram of an oxygen delivering system according to an embodiment of the present disclosure. The oxygen delivering system includes an oxygen provider 101, a controlling device 102 and a gas pathway 103.

It is noted that the gas pathway 103 may include a device gas pathway 103 (within the oxygen provider 101), a breathing circuit and a patient breathing interface.

With reference to FIG. 1, oxygen, which may be provided by an oxygen source, is input to the oxygen provider 101 where oxygen-containing gas is produced, and the oxygen-containing gas output by the oxygen provider 101, which is controlled by the controlling device 102, is delivered through the gas pathway 103 to the patient.

It is understood that, FIG. 1 is merely a logical schematic diagram of the oxygen delivering system, which shows an exemplary configuration of functional units. In a practical application scenario, the function units of the system may be implemented in various forms.

For example, the controlling device may be a compact device which is configured to implement the method provided in the present disclosure, for example, it may be a portable device which is connected between an oxygen provider and a patient, serving as a component of an oxygen delivering system which includes a plurality of components that may be assembled or connected by a caregiver onsite.

Or, the controlling device may be integrated into the oxygen provider as a hardware/software/firmware unit of the oxygen provider.

FIG. 2 is a schematic flowchart of a method for controlling oxygen-containing gas output by an oxygen provider according to an embodiment of the present disclosure. The method may be applied in the oxygen delivering system shown in FIG. 1. The method includes the following steps:

Step 201, a controlling device acquires a first pressure measurement of the oxygen-containing gas at the oxygen provider side.

The oxygen-containing gas is output by the oxygen provider through a device gas pathway. In a possible implementation, a pressure sensor is configured to detect the pressure level in the device gas pathway. The pressure sensor may be integrated in the oxygen provider.

In an oxygen therapy session, when the oxygen delivering system starts to operate, the oxygen-containing gas will be generated and delivered through the device gas pathway of the oxygen provider. During the process in which the oxygen-containing gas is being delivered via the device gas pathway, the controlling device acquires the first pressure measurement through the pressure sensor.

It should be understood that, the controlling device may acquire multiple pressure measurements, from which the first pressure measurement may be selected based on a preset screening criterion. For example, a pressure measurement within a preset range is determined as the first pressure measurement, and a pressure measurement out of the preset range is determined as an invalid pressure measurement.

Step 202, the controlling device determines a pressure estimation of the oxygen-containing gas at the patient side based on the first pressure measurement.

The oxygen-containing gas experiences a pressure drop on the gas pathway between the oxygen provider and a patient interface to arrive at the patient side. Therefore, it is necessary to determine a pressure level at the patient side.

In the embodiment, the pressure level at the patient side, which is referred to as a pressure estimation, is determined based on a pressure measurement at the oxygen provider side. The pressure estimation is calculated according to Equation 1.

P_{aw}=P_{machine}−ΔP Equation 1

Where P_{aw} is the pressure estimation of the oxygen-containing gas at the patient side, P_{machine} is the first pressure measurement, and ΔP is the pressure drop.

In a possible implementation, ΔP is determined based on a preset mapping relationship between a flow rate and a value of the pressure drop.

For example, a flow sensor is configured to detect the flow rate in the device gas pathway of the oxygen provider. The flow sensor may be integrated in the oxygen provider. The controlling device acquires a first flow measurement at the oxygen provider side through the flow sensor, and determines the value of ΔP based on the mapping relationship.

In another possible implementation, ΔP is determined based on the first flow measurement, and a characteristic property of the gas pathway from the oxygen provider side to the patient side. The characteristic property includes but not limited to a laminar resistance of the gas pathway or a turbulent resistance of the gas pathway.

For example, ΔP is calculated according to Equation 2.

ΔP=Rₗₐₘ·Q_{cir}+Rₜᵤᵣ*Q_{cir}² Equation 2

Where Rₗₐₘ is the laminar resistance of the gas pathway, Rₜᵤᵣ is the turbulent resistance of the gas pathway, and Q_{cir} is the first flow measurement.

It is noted that the characteristic property may be calculated according to an empirical formula which is derived from data obtained from experimental results, the method for calculating the characteristic property is not specifically limited in the present disclosure.

In a possible implementation, the characteristic property is determined based on a preset mapping relationship between a flow rate and a value of the characteristic property.

Step 203, the controlling device controls a target parameter of the oxygen-containing gas output by the oxygen provider based on the pressure estimation.

The target parameter of the oxygen-containing gas refers to a parameter that can be monitored and adjusted by the oxygen provider.

The controlling device adjusts the target parameter to guarantee that the pressure estimation of the oxygen-containing gas at the patient side satisfies a preset criterion.

For example, in an HFNC oxygen therapy session, the pressure estimation is required to be a positive pressure such that gas inhaled by a patient is from the oxygen-containing gas output by the oxygen provider rather than from the ambient environment.

The target parameter may include but not limited to a flow rate of the oxygen-containing gas.

In a possible implementation, the controlling device adjusts the flow rate of the oxygen-containing gas to guarantee that the pressure estimation of the oxygen-containing gas at the patient side is in a preset range [P_{minthreshold}, P_{maxthreshold}], where 0<P_{minthreshold}<P_{maxthreshold}.

Specifically, the controlling device may determine a flow setting of the oxygen provider based on the pressure estimation, and controls the flow rate of the oxygen-containing gas output by the oxygen provider based on the flow setting of the oxygen provider.

For example, the controlling device may apply a relatively small flow setting in the first place, so the value of the pressure estimation will be smaller than P_{minthreshold} then the controlling device gradually increases the flow setting by a preset amount each time, and keeps monitoring a current value of the pressure estimation, when the current value of the pressure estimation is within the preset range the controlling device maintains a steady flow setting unless the pressure estimation is out of the preset range. If the pressure estimation is smaller than P_{minthreshold}, the controlling device increases the flow setting by a preset amount. If the pressure estimation is greater than P_{maxthreshold}, the controlling device decreases the flow setting by a preset amount.

According to the method for controlling oxygen-containing gas output by an oxygen provider explained in the embodiments of the present disclosure, the pressure estimation of the oxygen-containing gas at the patient side is determined based on the first pressure measurement of the oxygen-containing gas at the oxygen provider side, and the oxygen-containing gas output by the oxygen provider is controlled based on the pressure estimation. Therefore, an automated solution to control the oxygen-containing gas based on a pressure measurement (the first pressure measurement) at the oxygen provider side is provided.

Comparing with the conventional oxygen therapy where the flow rate delivered to a patient is fixed, the target parameter of the oxygen-containing gas is monitored and adjusted based on the pressure estimation. Since the value of the first pressure measurement may be contingent on various factors, such as disease severity, age of the patient, and a status of the patient (sleep or awake), different values of the first pressure measurement may be acquired in different time with respect to a same patient, or with respect to different patients, consequently, different pressure estimations may be acquired due to these various factors, and therefore, the target parameter of the oxygen-containing gas will be adjusted to deferent levels. When the steps of the method are executed repeatedly, the target parameter of the oxygen-containing gas will be adjusted dynamically and adaptively on a case by case basis.

Furthermore, since the first pressure measurement of the oxygen-containing gas is acquired at the oxygen provider side, a pressure sensor for detecting the first pressure measurement may be configured at the oxygen provider side, for example, it may be connected to the oxygen provider or integrated in the oxygen provider, therefore, a pressure sensor at a patient side (typically attached to a patient) is no longer required, therefore, the oxygen delivering system may be designed as a more compact product and the complexity of a medical device for oxygen therapy is reduced, and the cost is therefore reduced.

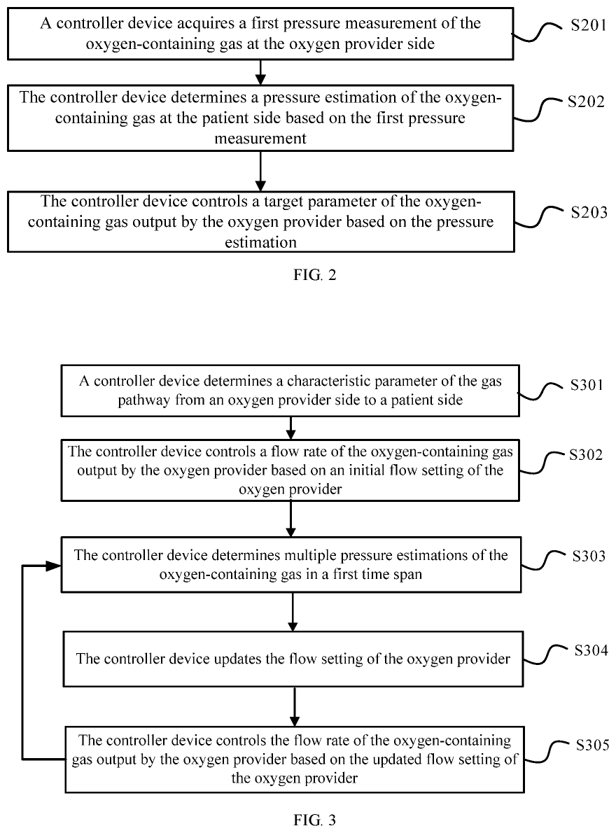

FIG. 3 is a schematic flowchart of a method for controlling oxygen-containing gas output by an oxygen provider according to an embodiment of the present disclosure.

In the embodiment, the method will be further explained in conjunction with an application scenario in an HFNC oxygen therapy session.

In an HFNC oxygen therapy session, a positive pressure is required to be maintained in a nasal cavity or a respiratory tract of a patient. Therefore, in the embodiment, the pressure estimation is required to be a positive pressure to guarantee that gas inhaled by a patient is from the oxygen-containing gas output by the oxygen provider rather than from the ambient environment.

The controlling device may work in a calibration mode and an operating mode. In the calibration mode, the characteristic property of the gas pathway is determined, and in the operating mode, the characteristic property is used to determine a pressure estimation at the patient side.

The method includes the steps as described in the following. Some of the steps which have already been explained in the embodiment corresponding to FIG. 2 will not be elaborated again for conciseness. The method may be applied in the oxygen delivering system shown in FIG. 1.

Step 301, a controlling device determines a characteristic property of the gas pathway from an oxygen provider side to a patient side.

In a possible implementation, the characteristic property of the gas pathway is determined in a case where a pressure at the patient side is zero. For example, in situation where the patient interface is not attached to a patient (or anything else), the pressure at the patient interface is considered as zero. Therefore, Step 301 may be executed before the patient interface is attached to a patient.

For example, a caregiver may assemble or connect the components of the oxygen delivering system on site and check that the gas pathway from the oxygen provider side to a patient side is not blocked, where the gas pathway include a device gas pathway (within the oxygen provider), a breathing circuit and a patient breathing interface.

Subsequently, the caregiver may switch the controlling device into a calibration mode. Before the controlling device finishes a calibration procedure, the caregiver may just hold the patient interface in the air.

In the calibration mode, the controlling device acquires a set of second pressure measurements at the oxygen provider side and a set of second flow measurements at the oxygen provider side, and determines the characteristic property based on the set of second pressure measurements and the set of second flow measurements.

For example, a pressure sensor and a flow sensor are integrated in the oxygen provider, the set of second pressure measurements and the set of second flow measurements are acquired through the pressure sensor and the flow sensor. According to Equation 1, in the case where a pressure at the patient side is zero, P_{aw}=0, therefore, ΔP=P_{machine}.

That is, in the case where a pressure at the patient side is zero, the oxygen-containing gas experiences a pressure drop (ΔP) that is equal to the second pressure measurements (P_{machine}), therefore, according to Equation 2, we have:

ΔP=P_{machine}=Rₗₐₘ·Q_{cir}+Rₜᵤᵣ*Q_{cir}² Equation 3

Taking the set of second pressure measurements as values of ΔP, taking the set of second flow measurements as values of Q_{cir}, the respective values of characteristic property Rₗₐₘ and Rₜᵤᵣ can be derived based on a curve fitting method.

If the respective values of Rₗₐₘ, and Rₜᵤᵣ are within a predetermined range, the controlling device determines that the values are valid, and the calibration procedure is finished.

If the respective values of Rₗₐₘ, and Rₜᵤᵣ are not within the predetermined range, the controlling device may repeat the calibration procedure or output warning information.

Step 302, the controlling device controls a flow rate of the oxygen-containing gas output by the oxygen provider based on an initial flow setting of the oxygen provider.

When the calibration procedure is finished, the controlling device may switch into an operating mode. The caregiver may now attach the patient interface to a patient.

In the operating mode, the controlling device may determine an initial flow rate of the oxygen-containing gas output by the oxygen provider, and controls the flow rate of the oxygen-containing gas output by the oxygen provider based on the initial flow setting.

The initial flow setting may be a preset value, or a value input by a user, or determined according to a preset mapping relationship between a value of flow setting and a value of a characteristic property, where the mapping relationship is preset based on data obtained from experimental results, in principle, the bigger the pressure drop induced by the gas pathway, the bigger the initial flow setting is.

Step 303, the controlling device determines multiple pressure estimations of the oxygen-containing gas in a first time span.

With reference to steps 201 and 202 in the embodiment corresponding to FIG. 2, the controlling device may acquire a first pressure measurement, determine a pressure drop ΔP based on the characteristic property and the first flow measurement, and determine a pressure estimation based on the first pressure measurement and the pressure drop ΔP.

Similarly, the controlling device may determine multiple pressure measurements based on multiple pressure measurements acquired in the first time span.

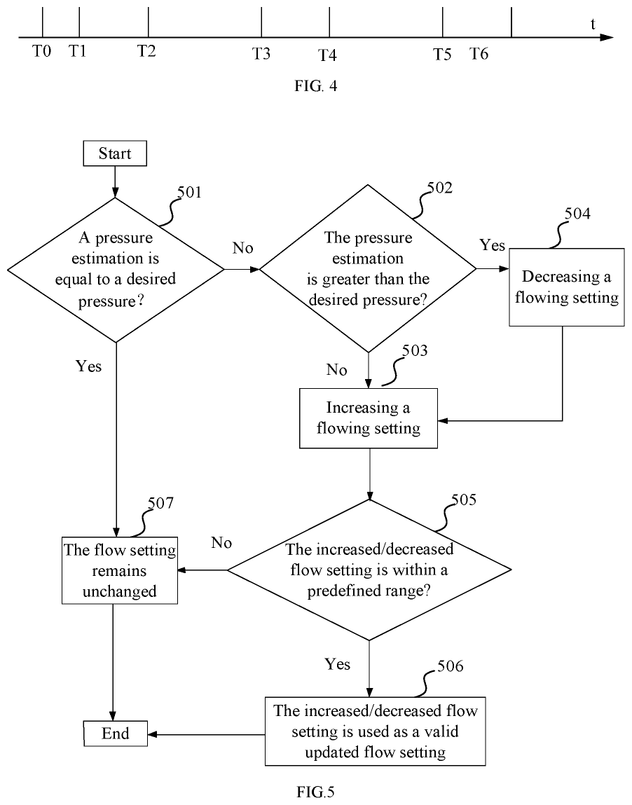

With reference to FIG. 4, the first time span may be a time span when the controlling device is working in the operating mode. For example, the controlling device switches to the operating mode at time T0, the first time span is a time span during time T1 and T2, which will be denoted as time span [T1, T2], where T1 is after T0, for example 5 seconds after T0.

The length of time span [T1, T2] may be preset, or it may be determined by the controlling device based on a breathing period (a time span for a breathing in and out process) of a patient.

The controlling device may acquire some physiological parameters of the patient, and determines the breathing period based on practical experiences or methods already known to a person skilled in the art, which will not be elaborated herein. In a possible implementation, the length of time span [T1, T2] equals to at least one breathing period.

It is noted that there may be multiple first time spans, with reference to FIG. 4, each of time spans [T1, T2], [T3, T4] and [T5, T6] is a first time span described herein. That is, step 303 may be executed repeatedly in different time spans.

Step 304, the controlling device updates the flow setting of the oxygen provider.

In the embodiment, the controlling device updates the flow setting dynamically based on a pressure estimation of the oxygen-containing gas at the patient side.

FIG. 5 is a schematic diagram for updating the flow setting of the oxygen provider according to an embodiment of the present disclosure. In block 501, a determination is made whether the pressure estimation of the oxygen-containing gas at the patient side is equal to a desired pressure.

If the pressure estimation is equal to the desired pressure, then the flow setting remains unchanged.

If the pressure estimation is not equal to the desired pressure, then in block 502, a determination is made whether the pressure estimation of the oxygen-containing gas at the patient side is greater than the desired pressure.

If the pressure estimation of the oxygen-containing gas at the patient side is not greater than the desired pressure, then in block 503, the flow setting of the oxygen provider is increased by a preset amount.

If the pressure estimation of the oxygen-containing gas at the patient side is greater than the desired pressure, then in block 504, the flow setting of the oxygen provider is decreased by the preset amount.

The flow setting of the oxygen provider is limited within a predefined range, in the block 505, a determination is made whether the increased/decreased flow setting is within the predefined range.

If the increased/decreased flow setting is within the predefined range, then in block 506, the increased/decreased flow setting is used as a valid updated flow setting.

If the increased/decreased flow setting is not within the predefined range, then in block 507, the flow setting remains unchanged.

In a possible implementation, the controlling device calculates a mean pressure of the multiple pressure estimations in a first time span, and update the flow setting of the oxygen provider based on the mean pressure in a second time span following the first time span.

For example, with reference to FIG. 4, the controlling device calculates a mean pressure of the multiple pressure estimations acquired in time span [T1, T2], and update the flow setting in [T2, T3].

Specifically, the controlling device increases the flow setting of the oxygen provider by a preset amount in time span [T2, T3] in response to that the mean pressure in time span [T1, T2] is lower than a desired pressure. Or, the controlling device decreases the flow setting of the oxygen provider by the preset amount in time span [T2, T3] in response to that the mean pressure in time span [T1, T2] is higher than the desired pressure.

Similarly, the flow setting may be updated in time span [T4, T5] based on a mean pressure of the multiple pressure estimations acquired in time span [T3, T4]

Step 305, the controlling device controls the flow rate of the oxygen-containing gas output by the oxygen provider based on the updated flow setting of the oxygen provider.

The controlling device controls the flow rate based on the flow setting, whenever the flow setting is updated, the controlling device adjusts the flow rate of the oxygen-containing gas output by the oxygen provider accordingly.

According to the method for controlling oxygen-containing gas output by an oxygen provider explained in the embodiments of the present disclosure, multiple pressure estimations of oxygen-containing gas at a patient side are determined based on multiple pressure measurements at the oxygen provider side, and the flow rate of the oxygen provider is adjusted based on the multiple pressure estimations.

Since the pressure measurements at the oxygen side may be contingent on various factors, different pressure estimations at the patient side may be acquired due to these various factors, consequently, the flow rate of the oxygen provider is adjusted dynamically and adaptively on a case by case basis.

Furthermore, a pressure estimation of the oxygen-containing gas at the patient side is determined based on a pressure measurement of the oxygen-containing gas at the oxygen provider side, therefore, a pressure sensor at a patient side (typically attached to a patient) is no long required, therefore, the oxygen delivering system may be designed as a more compact product and the complexity of a medical device for oxygen therapy is reduced.

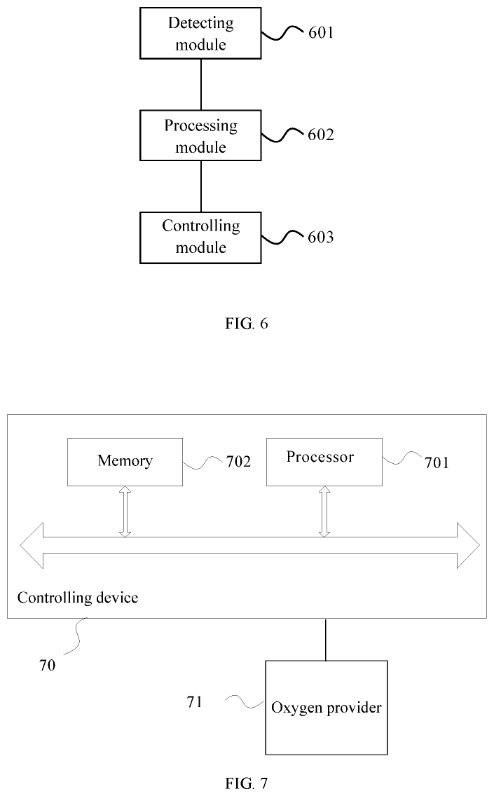

FIG. 6 is a schematic structural diagram of a controlling device according to an embodiment of the present disclosure. The controlling device, including:

a detecting module 601, configured to acquire a first pressure measurement of the oxygen-containing gas at the oxygen provider side;

a processing module 602, configured to determine a pressure estimation of the oxygen-containing gas at the patient side based on the first pressure measurement, where the oxygen-containing gas experiences a pressure drop on the gas pathway to arrive at the patient side; and

a controlling module 603, configured to control a target parameter of the oxygen-containing gas output by the oxygen provider based on the pressure estimation.

In a possible implementation,
the processing module 602 is further configured to determine a characteristic property of the gas pathway from the oxygen provider side to the patient side;

the detecting module 601 is further configured to acquire a first flow measurement at the oxygen provider side; and

the processing module 602 is further configured to determine the pressure estimation based on the characteristic property, the first flow measurement and the first pressure measurement.

In a possible implementation,

the detecting module 601 is further configured to acquire a set of second pressure measurements at the oxygen provider side and a set of second flow measurements at the oxygen provider side in a case where a pressure at the patient side is zero; and

the processing module 602 is further configured to determine the characteristic property based on the set of second pressure measurements and the set of second flow measurements.

In a possible implementation,

the processing module 602 is further configured to determine the pressure drop based on the characteristic property and the first flow measurement; and determine the pressure estimation based on the first pressure measurement and the pressure drop.

In a possible implementation,

the processing module 602 is further configured to determine a flow setting of the oxygen provider based on the pressure estimation; and

the controlling module 603 is further configured to control the flow rate of the oxygen-containing gas output by the oxygen provider based on the flow setting of the oxygen provider.

In a possible implementation, where the processing module 602 is configured to:

determine multiple pressure estimations of the oxygen-containing gas in a first time span; calculate a mean pressure of the determined multiple pressure estimations; and

in a second time span following the first time span, increase the flow setting of the oxygen provider in response to that the mean pressure is lower than a desired pressure; or

in the second time span following the first time span, decrease the flow setting of the oxygen provider in response to that the mean pressure is higher than the desired pressure.

FIG. 7 is a schematic structural diagram of a controlling device 70 according to an embodiment of the present disclosure. The controlling device 70 is communicatively connected to an oxygen provider 71, the oxygen provider 71 being capable of delivering the oxygen-containing gas via a gas pathway from the oxygen provider 71 side to a patient side, and the controlling device 70 includes:

at least one processor 701; and

a memory 702 communicatively connected with the at least one processor 701; where,

the memory 702 stores instructions executable by the at least one processor 701, and the instructions, when executed by the at least one processor 701, cause the at least one processor 701 to implement the method for controlling an oxygen provider according to the embodiments of the present disclosure.

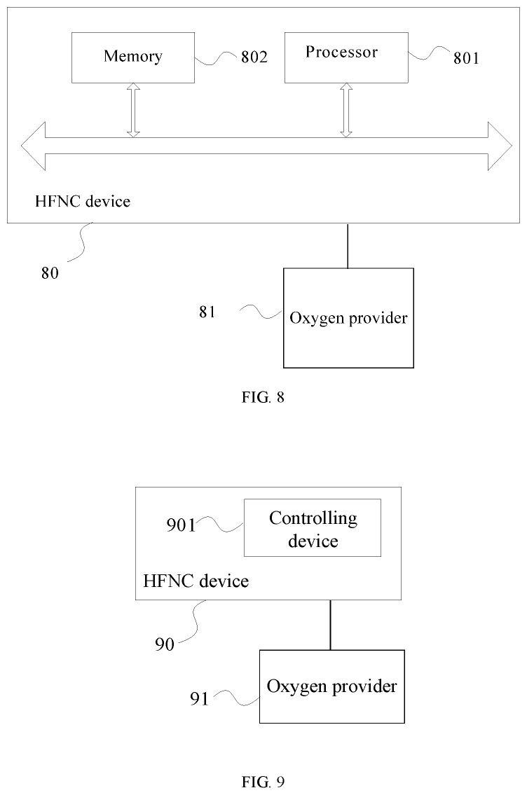

FIG. 8 is a schematic diagram of a high flow nasal cannula (HFNC) device 80 according to an embodiment of the present disclosure. The HFNC device 80 is communicatively connected to an oxygen provider 81, the oxygen provider 81 being capable of delivering the oxygen-containing gas via a gas pathway from the oxygen provider 81 side to a patient side, and the HFNC device 80 includes:

at least one processor 801; and

a memory 802 communicatively connected with the at least one processor 801; where,

the memory 802 stores instructions executable by the at least one processor 801, and the instructions, when executed by the at least one processor 801, cause the at least one processor 801 to implement the method for controlling an oxygen provider according to the embodiments of the present disclosure.

FIG. 9 is a schematic diagram of a HFNC device 90 according to an embodiment of the present disclosure. The HFNC device 90 is communicatively connected to an oxygen provider 91, the oxygen provider 91 being capable of delivering the oxygen-containing gas via a gas pathway from the oxygen provider 91 side to a patient side, and the HFNC device 90 includes a controlling device 901 according to the embodiment of the present application.

The present disclosure also provides a computer readable storage medium, storing thereon computer executable instructions which, when being executed by a processor, implement the method for controlling an oxygen provider according to embodiments of the present disclosure.

Terms such as “first”, “second” and the like in the specification and claims of the present disclosure as well as in the above drawings are intended to distinguish different objects, but not intended to define a particular order.

The term “a” or “an” is not intended to specify one or a single element, instead, it may be used to represent a plurality of elements where appropriate.

In one or more examples, the functions described may be implemented in hardware, software, firmware, or any combination thereof. For example, the functions may be implemented by one or more processors, such as one or more application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, the techniques could be fully implemented in one or more circuits or logic elements.

In the claims, the word “including” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate, preclude or suggest that a combination of these measures cannot be used to advantage.

The foregoing detailed description has been presented for purposes of illustration and description. It is not intended to be exhaustive or to limit the subject matter claimed herein to the precise form(s) disclosed. Many modifications and variations are possible in light of the above teachings. The described embodiments were chosen in order to best explain the principles of the disclosed technology and its practical application to thereby enable others skilled in the art to best utilize the technology in various embodiments and with various modifications as are suited to the particular use contemplated. Those embodiments with various modifications are within the range and scope of the following claims.

1. A method for controlling oxygen-containing gas output by an oxygen provider, the oxygen provider being capable of delivering the oxygen-containing gas via a gas pathway from the oxygen provider side to a patient side, the method comprising:
acquiring a first pressure measurement of the oxygen-containing gas at the oxygen provider side;
determining a pressure estimation of the oxygen-containing gas at the patient side based on the first pressure measurement, wherein the oxygen-containing gas experiences a pressure drop on the gas pathway to arrive at the patient side;
determining a set value for flow rate of the oxygen provider based on the pressure estimation; and
controlling a flow rate of the oxygen-containing gas output by the oxygen provider based on the set value for flow rate of the oxygen provider;
wherein determining the pressure estimation of the oxygen-containing gas at the patient side based on the first pressure measurement, comprises:
determining multiple pressure estimations of the oxygen-containing gas in a first time span;
wherein before controlling the flow rate of the oxygen-containing gas output by the oxygen provider based on the set value for flow rate of the oxygen provider, the method further comprises:
calculating a mean pressure of the determined multiple pressure estimations;
wherein determining the set value for flow rate of the oxygen provider based on the pressure estimation, comprises:
in a second time span following the first time span, increasing the set value for flow rate of the oxygen provider in response to the mean pressure being lower than a desired pressure; or
in the second time span following the first time span, decreasing the set value for flow rate of the oxygen provider in response to the mean pressure being higher than the desired pressure;
wherein before determining the pressure estimation of the oxygen-containing gas at the patient side based on the first pressure measurement, the method further comprises:
determining a characteristic property of the gas pathway from the oxygen provider side to the patient side; and
controlling the flow rate of the oxygen-containing gas output by the oxygen provider based on an initial flow setting of the oxygen provider, wherein the initial flow setting is determined according to a preset mapping relationship between a value of flow setting and a value of the characteristic property.

2. The method according to claim 1, wherein determining the pressure estimation of the oxygen-containing gas at the patient side based on the first pressure measurement, comprises:
acquiring a first flow measurement at the oxygen provider side; and
determining the pressure estimation based on the characteristic property, the first flow measurement and the first pressure measurement.

3. The method according to claim 1, wherein determining the characteristic property of the gas pathway from the oxygen provider side to the patient side, comprises:
acquiring a set of second pressure measurements at the oxygen provider side and a set of second flow measurements at the oxygen provider side in a case where a pressure at the patient side is zero; and
determining the characteristic property based on the set of second pressure measurements and the set of second flow measurements.

4. The method according to claim 2, wherein determining the pressure estimation based on the characteristic property, the first flow measurement and the first pressure measurement, comprises:
determining the pressure drop based on the characteristic property and the first flow measurement; and
determining the pressure estimation based on the first pressure measurement and the pressure drop.

5-6. (canceled)

7. A controlling device, the controlling device is communicatively connected to an oxygen provider, the oxygen provider being capable of delivering oxygen-containing gas via a gas pathway from the oxygen provider side to a patient side, and the controlling device comprises:
at least one processor; and
a memory communicatively connected with the at least one processor; wherein,
the memory stores instructions executable by the at least one processor, and the instructions, when executed by the at least one processor, cause the at least one processor to:
acquire a first pressure measurement of the oxygen-containing gas at the oxygen provider side;
determine a pressure estimation of the oxygen-containing gas at the patient side based on the first pressure measurement, wherein the oxygen-containing gas experiences a pressure drop on the gas pathway to arrive at the patient side;
determine a set value for flow rate of the oxygen provider based on the pressure estimation; and
control a flow rate of the oxygen-containing gas output by the oxygen provider based on the set value for flow rate of the oxygen provider;
wherein the instructions further cause the at least one processor to:
determine multiple pressure estimations of the oxygen-containing gas in a first time span;
calculate a mean pressure of the determined multiple pressure estimations; and
in a second time span following the first time span, increase the set value for flow rate of the oxygen provider in response to the mean pressure being lower than a desired pressure; or, in the second time span following the first time span, decrease the set value for flow rate of the oxygen provider in response to the mean pressure being higher than the desired pressure;
wherein the instructions further cause the at least one processor to:
determine a characteristic property of the gas pathway from the oxygen provider side to the patient side, wherein the characteristic property is determined based on a preset mapping relationship between a flow rate and a value of the characteristic property; and
control the flow rate of the oxygen-containing gas output by the oxygen provider based on an initial flow setting of the oxygen provider, wherein the initial flow setting is determined according to a preset mapping relationship between a value of flow setting and a value of the characteristic property.

8-12. (canceled)

13. A high flow nasal cannula (HFNC) device, the HFNC device is communicatively connected to an oxygen provider, the oxygen provider being capable of delivering the oxygen-containing gas via a gas pathway from the oxygen provider side to a patient side, wherein the HFNC device comprises a controlling device according to claim 7.

14. A non-transitory computer readable storage medium, storing thereon computer executable instructions which, when being executed by a processor, implement a method for controlling oxygen-containing gas output by an oxygen provider according to claim 1.

15. The method according to claim 1, wherein the characteristic property is determined based on a preset mapping relationship between a flow rate and a value of the characteristic property.
